SEQUENCE LISTING (7 sequences)

SEQ ID NO: 1 - DNA, 491 bp, Salmo salar
  mRNA (1)..(491): mRNA sequence originating from cDNA cloning.
agtctctctctccaagcagcctcagacaaaaccagaccaacagtgttcacttcagcaaccgactgctcctaacatgtgagggcaggtatgtctttcacgtccaggacttcaagaacaacatggcccagatccctactaacagcagtctggccctgtcctacccctgtcctgaaggccagtatgactttcgctttgtggtgagaccagaatacatctgatagctctgcctggcacagatgcaacaacgctcctaacacacatccacaaatctaataatgatgtcccattgccaatcaattgctaatttcttttattcaagagtttgaaattatggtaatgtggttttagaaatatgtgactgatttgatcataaccctatgttgatttgctgaaagcatatcaacatgtagcactgctcttatgtaagtattaatcaaaaactataacatctctgcttgcacttgaataaaggcatttgaacaagaaaaaaa

SEQ ID NO: 2 - DNA, 425 bp, Salmo salar
  mRNA (1)..(425): mRNA sequence originating from cDNA cloning.
agcaacattgaagcatgacttttcagcaacccttcttcaatccgagagtgccgttcactggctgtatccagggagctctgcatgaggggaagaccatcactgtgacagggagagtcctgccaggagcccagaggtttcatgtgaatttgcaatgtggctcaagaggaaatcctgacatagccctccacttcaacccccgatatgacagcttcctagacgttgtgatctgcaacaccatgcagcactccaagtggggttcagaggagcgtgaatactttgcacccatgacccggggggccaactttaccctcatgttcctggtcaacaaagattcatattcgattattgtgaatggtgccctcttcatggagtacctgcaccggctctcgttctccagagtggacaccatctctgtggatggggga

SEQ ID NO: 3 - DNA, 680 bp, Salmo salar
  mRNA (1)..(680): mRNA sequence originating from cDNA cloning.
aagcactaaaatggctgacaatcaggaactgttctgttgctccgtctgtctggatctactgaaggatccggtgactactgcctgtggacacagttactgtatgggctgtattaaagaaagctgggatcaggatgatctgaaaggtgtctacagatgtccccagtgcagacagacctttaccccaaggcctgttctgaagagaaacaccgtgctggctgaagtgttggagaatttgaaaatggcagatttcaaaggtaccactcctgcacactgttatgctgaacctgaagatgtggagtgtgatgtctgcactaggaaaaaactcaaagctgtcaagtcctgtctggtgtgtctggcctcttactgtgagactcatctccagcctcactactatgtgtccccactaaagaagcataagcttgtcaatgcagtgacagatctacaggagaagatttgcccaaaccatgacaaactgctggaggtttactgtcgtaccgatcagcagtgtatctgtctgctgtgtttgatggatgaacataaaggccatgatacagtgtcagctgcagcagagaggactgagaaacagcaacagttgggagaaacacagaaggaagggaagcagagagcccagaggaaagagaaggaggtgcaggagctgagacaggctgtagatgccatta

SEQ ID NO: 4 - DNA, 799 bp, Salmo salar
  mRNA (1)..(799): mRNA sequence originating from cDNA cloning.
gggggccaactttaccctcatgttcctggtcaacagagattcatattcgctgattgtgaatggtgctcacttcatggagtacctgcaccggctgtcgttctccagagtggacaccatcaccgtggatgggggagtggagatccaatccattgccttctccaaccctgcagtgacatcacctcctccccaaccaggatatcctgtccatccacacagtggtcaacggaaccgaaccaaatctcatcaaagtaaatcatgtcccaaaactcctcctcaatggtgtaatgccacacaggctttctcggctgcgttttgcgccccaggactttttcagacaccccctccatacaccccccagccgtccttcgttgtgccatataaaaatataatggtaggtggactttacccaggcagaaacatcatcgtccagggtgtggtcaaccacaacgcttataggttctatgttaacctgcgtttcaactcgggggtggcgctccacttcaaccccaggtttgatgagaacactgtcgtgcgtaatagtctcctgaaggaacagtggggtccagaggagaggaaagggggcatgcccttctacagaggccagccttttacgatgaccattacgtgtgacaccaagtgctacagggtgatggtgaatggagtccagatgttcagctacaaccaccgccacttcctgctccagcagatagatatcctggaggtggaaggggacgtgagcctgtcctctgtggtgttctaagtaatgactctgtcctctgttatggtttgagtcacatac

SEQ ID NO: 5 - DNA, 673 bp, Salmo salar
  mRNA (1)..(673): mRNA sequence originating from cDNA cloning.
gntgggggagtggaagtcctgtctattgccttctccaaccctgcagtcacctcacctcctccccaaccaggatatcctggccatccacacagtggtcaacgaaaccgaaatagatctcgtcaaagcaaaactcatcaacccaaaactcctcctcaatggtgtaatgccacacaggctttctcagctgcaggatttcttcaggcaccccctccatacactgcccagcctccatacacccccaagccatcctttgttgtgccgtataaaaacataatggctggtggactttacccaggcagaaacatcaccatccagggtgtggtcaacccaacgctgataagttctgtatcaacctgcgtttcaactcgggggtggcgttccacttcaaccccaggttcaatgagaacgttgtagttcgtaacagtctcctgaaggaacagtggggtccagaggaaaggacaggtggcatgcccttctacagaggccaacctcttacggtgatcatcatgtgtgatacccaatgctacagggtgatggtgaatggagccctgatgttcagctacaaccaccgccacttccttttccagcagattgacattctggaggtggagggggacgtgagcctgtccgctgtgctggtctgagttacaacacattgaggtcaaagttcagc

SEQ ID NO: 6 - DNA, 832 bp, Salmo salar
  mRNA (1)..(832): mRNA sequence originating from cDNA cloning.
tcaatatcaaagattatgcagactgtctctgcggacccctgggagattacgcgcagctctataaaactggggaggcgcctgggcgaaggtttctttggagtggtctatcaaggcctctataacaacactccagttgcagtgaagacccttagacatggcaccatggaccctcgggacttcctaaaagaggcccagatcatgaagactatggagcatcctaacctcatccagctcttggctgtctgcactgaagaacccatctacatcatcactgagctaatgaagaacggaagtctgctggattacctgaaggtcagggttggaaggctacatttaaatgaccagattgagatggcagcccaagtggcttctgggatggcttatctggagatgaagaaatacatccacagggacctggcagccaggaatgtgctggtcggcgagaacaacgtctacaaggtggctgactttggccttgccagggtcttacaagccccaccgaatcagaccaacctgtattatatacctgtagggaaagcgatattccctttgagatggacggctcctgaggccatcgccaacgagaagttcaccatcaagtgtgacgtgtggtccttcggaatcttgctgtatgagatcatgacctttggggagatgccctatccagacatgaacaactcccaggtgaaacagagggttcccaatgggtacaggatgcctcgccctgttgacccctactgtttccaagacatgtacaagatcatgttggactgctggaatgggaataaaaatgacaggcccactttcgaggctctgaaaatgagtctgagga

SEQ ID NO: 7 - DNA, 731 bp, Salmo salar
  mRNA (1)..(731): mRNA sequence originating from cDNA cloning.
aaccatcacacaacggggaaatccttacttacatagcatcaataacagactaaatctacacttcacacacttcccttctccagaagccatcctcactcacgcatacacacacatatatatcttcagacacacagcttgagtccagtccagacccctcagtacagctcaggttatcggtgaccagtcatgatgcctaccgtgggtattgccaagatcctcttctgtgttctgttcctctacagctgctgccgagtgactcagggccagatggtgatggactgctgtctgacggtcagtcagagagcgatacctagacacgttgtcataggttaccagcctcaggtcagaggtcaaggctgctccatcagcgctgtggtcttcactaccaggaggggagtcaagctgtgtgctcccactgaccccacctgggttacagacctgatgaatctcatggaccgcctcatcaagaaatgccacgaaaccaacttcaaggctaaacactgtaagaagctgatgcacaagagtccctaaggtccctgtcagagcccctcctaccaccaccaccaccaccacctccacctccaccccccctggtcaaaacaaccatgacagagcctgagaagaaccttccaagtgggtgaagaacaggatgggagaagtaaaacaacgaacaaaaaagacnaatgtgattcctcttaccactaatgcacttttttattaagtttccttct

(19) United States
(12) Patent Application Publication
(10) Pub. No.: US 2009/0149641 A1
(43) Pub. Date: Jun. 11, 2009

(54) Fish disease protection

(75) Inventors: Sven Martin Jorgensen, Oslo (NO); Aleksei Krasnov, As (NO)

Correspondence Address: CHRISTIAN D. ABEL, ONSAGERS AS, POSTBOKS 6963 ST. OLAVS PLASS, NORWAY, N-0130, NO

(73) Assignee: NOFIMA AKVAFORSK-FISKERIFORSKNING AS, As (NO)

(21) Appl. No.: 12/042,356

(22) Filed: Mar. 5, 2008

Related U.S. Application Data
(60) Provisional application No. 60/893,383, filed on Mar. 7, 2007.

(51) Int. Cl. C07H 21/02

(52) U.S. Cl. 536/23.5

(57) ABSTRACT

The present invention concerns genes associated with viral disease in fish, particularly fish from the family Salmonidae, and more particularly Atlantic salmon.

### CROSS REFERENCE TO RELATED APPLICATIONS

This application claims the benefit under 35 USC §119(e) to U.S. provisional patent application 60/893,383, filed 7 Mar. 2007, the entire contents of which are hereby incorporated by reference.

### FIELD OF INVENTION

The present invention concerns genes associated with viral disease in fish, particularly fish from the family Salmonidae, and more particularly Atlantic salmon.

### BACKGROUND OF INVENTION

Current diagnostics of infectious diseases in aquaculture are to a large part based on detection of known pathogens after clinical signs of disease. This approach is insufficient for effective disease control due to high risk of both false positive and false negative results. Fish infected with protracted or non-pathogenic strains of bacteria and viruses do not develop disease while assays give positive results. Pathogenicity is explained by minor mutations or changes in their genetic composition and therefore development of assays for discrimination between pathogenic and non-pathogenic strains is time-consuming and expensive. Viruses that are already present in the farmed stocks generate mutated strains and this process is accelerated with high selection pressure for rapid propagation and spread among hosts. This can be exemplified by a well-characterised virus affecting aquaculture on the Northern hemisphere today; the infectious salmon anaemia virus (ISAV). The virus was first discovered in Norwegian aquaculture in 1984 and causes the severe infectious salmon anaemia disease resulting in high economical losses for affected farms. Today we see an escalation in new strains appearing and an increased confusion from both industry and scientists related to how to implement robust and reliable risk assessments for handling the virus/disease. According to the recent conclusions from an expert scientific committee (“Which risk factors relating to spread of Infectious Salmon Anaemia (ISA) require development of management strategies.” Opinion of the Panel on Animal Health and Welfare of the Norwegian Scientific Committee for Food Safety, 26 Jan. 2007, Dok.nr. 06/804) our knowledge is scarce when it comes to whether or not, and to which degree, different non-pathogenic strains pose a threat (if they give disease) and if action should be taken if such strains are detected in respective farms. So far, assays for pathogen detection must be constantly developed and updated. Situation is even harder and more dangerous when dealing with new fully unknown or poorly studied diseases. One such example is the newly discovered salmon alpha virus causing pancreas disease in an increasing number of geographically spread fish farms. Isolation and structural characterisation of new pathogens is tedious and time-consuming, and unless assays are developed there is no way for epidemiological control of new pathogens.

There is an explicit need for a simple and inexpensive test to decide if a fish infected with a pathogen develop clinical disease. To solve these problems, pathogen detection must be supplemented with assays to diagnose the disease status of the fish.

### SUMMARY OF THE INVENTION

The present invention provides a purified isolated mRNA of a viral-responsive gene derived from fish, particularly derived from fish of the family Salmonidae, and more particularly from Salmo salar.

### BRIEF DESCRIPTION OF THE DRAWINGS

FIG. 1 depicts the cumulative mortality in the control group.

FIG. 2 depicts the cumulative mortality of the fish in the infection trial.

### DETAILED DESCRIPTION OF THE INVENTION

Until recently, studies of interaction between fish and pathogen have been limited to a relatively small set of immune genes and proteins, such as interferons and cytokines of the innate arm of immunity and immunoglobulins and MHC (Major Histocompatibility Complex) of the adaptive arm of immunity. Recent advances in functional genomics have substantially expanded the possibilities to search for markers of the disease status.

A microarray platform for studies of fish of the Salmonidae family's response to pathogens and stressors was used. This microarray chip contains a comprehensive set of genes involved in immunity and immune-related functions, and among these genes with unknown function. In comparison of fish with high and low susceptibility to infectious salmon anemia virus (ISAV) a group of genes with high correlation between expression levels and severity of disease was identified. This tendency was observed in all studied tissues. These genes, were designated as VRG (Viral-Responsive Genes). The VRG was screened against an in-house gene expression database and it was found that VRG responded almost exclusively to viruses being insensitive to other stressors. It was also found that the VRG were up-regulated in rainbow trout infected with a completely different virus, namely the rhabdovirus viral haemorrhagic septicaemia virus (VHSV). The VRG group includes genes from different structural families, but the involvement of several VRG (e.g. galectin-like proteins) in viral responses have been suggested in mammals, however they have not been studied in fish. Other VRG products show only slight resemblance to the mammalian proteins with unknown functions.

These VRG may be used in the development of diagnostic assays for the disease status in fish caused by different viruses. From a structural study of VRG by sequence analyses of ˜500 000 Atlantic salmon ESTs (Expressed Sequence Tag), clusters for several VRG were found.

Information from genes that are specifically expressed in fish in response to development of symptomatic disease by the pathogen may represent a powerful source for the development of a diagnostic tool that will give a huge advantage over today's diagnostics based on detection of the pathogen itself. An exemplary comparison can be found in human clinical medicine where the diagnosis of any viral disease is based on CRP (C-Reactive Protein)-measurement which indicates an acute inflammation in the patient. Further development of a diagnostic assay for the detection of viral diseases in farmed salmon is outlined in the project description, (“Development of assay for diagnosis of viral diseases in farmed salmon: New tool for disease control based on host-pathogen interactions”), appended to U.S. provisional patent application 60/893,383, which is incorporated in its entirety herein by reference.

### EXAMPLE

### Experimental Virus Infection Trial

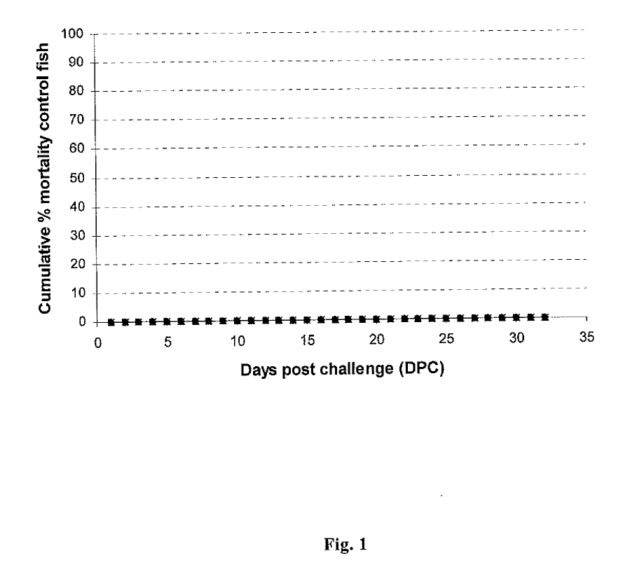

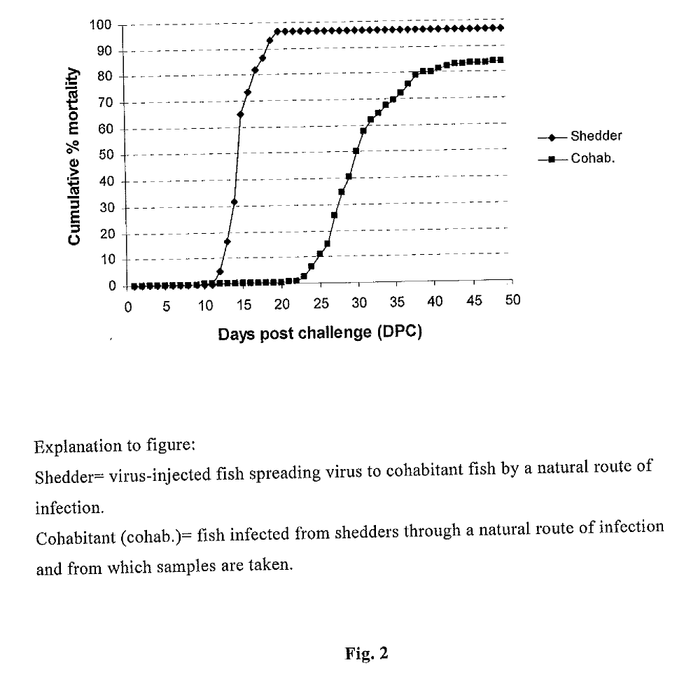

An infection trial was performed at the ISO-certified facilities of VESO Vikan. 360 unvaccinated and pathogen-free post-smolt Atlantic salmon (Salmo salar L.) from a genetically diverse population were infected with a pathogenic/acute-disease strain (Glesvaer/2/90 isolate) (Falk K, Namork E, Rimstad E, Mjaaland S, Dannevig B H. J Virol 1997, 71(12):9016-9023. PMID: 9371558) of infectious salmon anemia virus (ISAV, Orthomyxoviridae, genus Isavirus) by cohabitant exposure from intraperitoneal injected fish. Mortality was continuously recorded (84% cumulative mortality) and moribund fish were sampled from two extreme groups; the first 12 virus-susceptible fish and the last 12 virus-resistant fish (FIG. 2). In addition, control fish were sampled from each stage (FIG. 1). Tissue samples were taken from liver, heart, spleen and gills and stored in RNAlater reagent for subsequent extraction and purification of total RNA.

Microarray Detection of Novel Genes Associated with Viral Disease

Tissue samples were homogenised (IKA homogenizer) and total RNA was extracted using Trizol and silica membrane column-purification (PureLink, Invitrogen). Twenty microgram of pooled (n=4) and individual RNA samples (n=8) from the two stages were tested against control samples on a micro-array chip containing 1800 cDNA ESTs (Expressed Sequence Tags) (FA2.0 DNA microarray chip, University of Kupio, Finland) using a dye-swap or single-slide design with Cy3- and Cy5-dCTP labelling. Scanning and image processing of spots, subtraction of mean background and data normalization (Lowess) were performed as previously described (Krasnov A, Koskinen H, Pehkonen P, Rexroad C E 3rd, Afanasyev S, Molsa H. BMC Genomics 2005, 6(1):3.PMID: 15634361). Differentially expressed genes (Student's t-test, ANOVA, p>0.01) between resistant and susceptible fish were detected using an in-house software and database containing data from previous experiments based on the same micro-array platform. From the results of screening against ˜200 samples from different experiments, 7 genes, SEQ ID NOs. 1-7, were selected which were strongly up-regulated after infection in all tissues from susceptible fish compared to resistant fish.

1. A purified isolated mRNA of a viral-responsive gene derived from fish.

2. The mRNA as defined in claim 1 derived from a salmonide.

3. The mRNA as defined in claim 2 derived from Salmo salar.

4. The mRNA as defined in claim 3 wherein said nucleic acid is selected from SEQ ID NO 1, 2, 3, 4, 5, 6 and 7, sequences-conservative variants thereof, and functional-conservative variants thereof.

5. An isolated nucleic acid comprising a sequence selected from SEQ ID NO 1, 2, 3, 4, 5, 6 and 7, or degenerate variants thereof.

6. An isolated nucleic acid comprising a sequence that is at least 80% identical to a sequence selected from SEQ ID NO 1, 2, 3, 4, 5, 6 and 7.

7. An isolated nucleic acid according to claim 6, wherein the sequence is at least 90% identical to a sequence selected from SEQ ID NO 1, 2, 3, 4, 5, 6 and 7.

8. An isolated nucleic acid according to claim 7, wherein the sequence is at least 95% identical to a sequence selected from SEQ ID NO 1, 2, 3, 4, 5, 6 and 7.
